(19) United States
(12) Patent Application Publication
(10) Pub. No.: US 2017/0007633 A1
(43) Pub. Date: Jan. 12, 2017

(54) TREATMENT OF NEURODEGENERATIVE AND NEURODEVELOPMENTAL DISEASES BY INHIBITION OF THE a2-Na/K ATPase/a-ADDUCIN COMPLEX

(71) Applicant: PRESIDENT AND FELLOWS OF HARVARD COLLEGE, Cambridge, MA (US)

(72) Inventors: Azad Bonni, Richmond Heights, MO (US); Gilbert Gallardo, Maplewood, MO (US)

(21) Appl. No.: 15/117,917

(22) PCT Filed: Feb. 10, 2015
(86) PCT No.: PCT/US2015/015166

§ 371 Date: Aug. 10, 2016

Related U.S. Application Data
(60) Provisional application No. 61/937,871, filed on Feb. 10, 2014.

(51) Int. Cl. A61K 31/7048; G01N 33/50; G01N 33/68; C12N 15/113

(52) U.S. Cl. CPC A61K 31/7048; C12N 15/1137; C12N 15/1138; G01N 33/5058; G01N 33/6896; C12N 2310/14; C12N 2320/30; G01N 2800/28; G01N 2800/50; G01N 2333/914; G01N 2333/705

(57) ABSTRACT

Described herein are methods for the prevention of neurodegeneration and the treatment of neurodegenerative disease (Including amyotrophic lateral sclerosis) ami neurodevelopmental disorders through the administration of an agent that inhibits die a2-Na/K ATPase/a-Adducin Complex.

### RELATED APPLICATIONS

This application claims the benefit of priority to U.S. Provisional Patent Application Ser. No. 61/937,871, filed Feb. 10, 2014, which is hereby incorporated by reference in its entirety.

### GOVERNMENT INTEREST

This invention was made with Government support under National Institutes of Health Grant NRSA T32 5T32AG00222-17. The Government has certain rights in the invention.

### BACKGROUND

Neurodegenerative diseases are characterized by a progressive neurodegenerative process in which neuron structure and/or function is lost over time. Among the most common and most severe neurodegenerative diseases are amyotrophic lateral sclerosis (ALS), Alzheimer's disease, Parkinson's disease and Huntington's disease. Though genetic characteristics have been linked with some neurodegenerative diseases, such as the Huntingtin gene in Huntington's disease, for most neurodegenerative diseases the cause remains unclear. What is more, effective treatments have proven elusive for nearly all forms of neurodegenerative disease.

Astrocytes represent the most abundant cell type in the central nervous system (CNS) and have diverse functions in the developing and mature CNS. Astrocytes and neurons share a common lineage during development, and often both cell types express disease genes that trigger neurodegeneration in the CNS. Astrocytes are beginning to emerge as critical targets of CNS disorders that were once thought to selectively afflict neurons and mounting evidence suggests that astrocytes play a fundamental role in the progression of many neurodegenerative diseases. For example, expression of mutant proteins in astrocytes an ALS, Huntington's disease, and spinocerebellar ataxias induce non-cell autonomous neurodegeneration. However with few exceptions, the cell intrinsic mechanisms operating in mutant astrocytes that trigger non-cell autonomous neurodegeneration remain largely unknown.

ALS (also known as Lou Gehrig's disease), is a neurodegenerative disease characterized by the progressive degradation of motor neurons, which causes the afflicted individual to experience progressive weakness, muscle atrophy and respiratory compromise. ALS is the most common motor neuron disease in adults and is characteristically fatal within five years of onset. Currently, ALS has no cure and the only FDA approved treatment for ALS, the sodium channel blocker Riluzole, only increases patient survival by 2-3 months on average.

Thus, there is a need for new and improved compositions and methods for the treatment of neurodegenerative diseases, including ALS.

### SUMMARY

Described herein are methods for reducing neurodegeneration and/or treating or preventing a neurodegenerative disease (e.g., ALS, Huntington's disease, Alzheimer's disease, Parkinson's disease, spinal muscular atrophy (SMA), primary lateral sclerosis (PLS), progressive muscular atrophy (PMA), traumatic brain injury, spinocerebellar ataxias and/or progressive bulbar palsy (PBP)) in a subject through the inhibition of α2-Na/K ATPase and/or α-Adducin. Also described herein are methods of treating neurodevelopmental disorders (e.g., fragile X syndrome, Down's syndrome, Rett syndrome, intellectual disability, autism or an autism spectrum disorders such as Asperger syndrome) through the inhibition of α2-Na/K ATPase and/or α-Adducin.

In some aspects, provided herein is a method of treating a neurodegenerative disease or a neurodevelopmental disorder that includes administering to a subject an agent that inhibits α2-Na/K ATPase. In some embodiments, the neurodegenerative disease is ALS, Huntington's disease, Alzheimer's disease, Parkinson's disease, SMA, PLS, PMA, traumatic brain injury, spinocerebellar ataxias or PBP. In some embodiments, the neurodegenerative disease is ALS. In some embodiments, the neurodevelopmental disorder is fragile X syndrome, Down's syndrome, Rett syndrome, intellectual disability, autism or an autism spectrum disorders such as Asperger syndrome.

In some embodiments, the agent that inhibits α2-Na/K ATPase is a small molecule. In some embodiments, the small molecule is a cardiac glycoside. In some embodiments, the agent is digoxin, ouabain, digitoxin, proscillaridin A, digoxigenin, gitoxin, gitoxigenin, oleandrin, butalin, cinobufagenin, UNBS1450 or lanatoside C. In some embodiments, the agent is digoxin.

In some embodiments, the agent that inhibits α2-Na/K ATPase is an interfering nucleic acid molecule specific for α2-Na/K ATPase. In some embodiments, the interfering nucleic acid molecule is an antisense molecule, an siRNA molecule, an shRNA molecule or a miRNA molecule.

In some embodiments, the agent that inhibits α2-Na/K ATPase is an antibody that binds to α2-Na/K ATPase. In some embodiments, the antibody binds to an extracellular epitope of α2-Na/K ATPase. In some embodiments, the antibody is monoclonal or polyclonal. In some embodiments, the antibody is chimeric, humanized or human. In some embodiments, the antibody is a full length immunoglobulin molecule. In some embodiments, the antibody is an scFv, a Fab fragment, a Fab′ fragment, a F(ab′)2, a Fv, a NANOBODY® or a disulfide linked Fv.

In some embodiments, the agent inhibits the formation of a complex between α2-Na/K ATPase and α-Adducin. In some embodiments, the agent is an isolated soluble polypeptide comprising at least 5 consecutive amino acids of the amino acid sequence encoding α-Adducin.

In some embodiments, the method includes the step of administering a second agent. In some embodiments, the second agent is a therapeutic agent for the treatment of a neurodegenerative disease. In some embodiment, the second agent is an agent that inhibits α-Adducin.

In some aspects, provided herein is a method of treating a neurodegenerative disease or a neurodevelopmental disorder that includes administering to a subject an agent that inhibits α-Adducin. In some embodiments, the neurodegenerative disease is ALS, Huntington's disease, Alzheimer's disease, Parkinson's disease, SMA, PLS, PMA, traumatic brain injury, spinocerebellar ataxias or PBP. In some embodiments, the neurodegenerative disease is ALS. In some embodiments, the neurodevelopmental disorder is fragile X syndrome, Down's syndrome, Rett syndrome, intellectual disability, autism or an autism spectrum disorders such as Asperger syndrome.

In some embodiments, the agent that inhibits α-Adducin is a small molecule, an antibody, an interfering nucleic acid or a polypeptide. In some embodiments, the agent that inhibits α-Adducin is an interfering nucleic acid molecule specific for α-Adducin. In some embodiments, the interfering nucleic acid molecule is an antisense molecule, an siRNA molecule, an shRNA molecule or a miRNA molecule.

In some embodiments, the agent inhibits the formation of a complex between α2-Na/K ATPase and α-Adducin. In some embodiments, the agent is an isolated soluble polypeptide comprising at least 5 consecutive amino acids of the amino acid sequence encoding α2-Na/K ATPase.

In some embodiments, the method includes the step of administering a second agent. In some embodiments, the second agent is a therapeutic agent for the treatment of a neurodegenerative disease. In some embodiment, the second agent is an agent that inhibits α2-Na/K ATPase.

In certain aspects, provided herein is a method of determining whether a test agent is a candidate therapeutic agent for the treatment of a neurodegenerative disease (e.g., ALS, Huntington's disease, Alzheimer's disease, Parkinson's disease, SMA, PLS, PMA, traumatic brain injury, spinocerebellar ataxias or PBP) or a neurodevelopmental disorder (e.g., fragile X syndrome, Down's syndrome, Rett syndrome, intellectual disability, autism or an autism spectrum disorders such as Asperger syndrome). In some embodiments, the method includes: a) forming a test reaction mixture comprising: a α2-Na/K ATPase polypeptide or fragment thereof; a α-Adducin polypeptide or fragment thereof; and a test agent; b) incubating the test reaction mixture under conditions conducive for the formation of a complex between the α2-Na/K ATPase polypeptide or fragment thereof and the α-Adducin polypeptide or fragment thereof; and c) determining the amount of the complex in the test reaction mixture. In some embodiments, a test agent that reduces the amount of the complex in the test reaction mixture compared to the amount of the complex in a control reaction mixture is determined to be a candidate therapeutic agent for the treatment of a neurodegenerative disease or a neurodevelopmental disorder. In some embodiments, the test agent is an antibody, a protein, a peptide or a small molecule. In some embodiments, the test agent is a member of a library of test agents.

In certain embodiments, the control reaction mixture is substantially identical to the test reaction mixture except that the control reaction mixture does not comprise a test agent. In some embodiments, the control reaction mixture is substantially identical to the test reaction mixture except that the control reaction mixture comprises a placebo agent instead of a test agent.

In some embodiments, the test reaction mixture is formed by adding the test agent to a mixture comprising the α2-Na/K ATPase polypeptide or fragment thereof and the α-Adducin polypeptide or fragment thereof. In certain embodiments, the test reaction mixture is formed by adding the α2-Na/K ATPase polypeptide or fragment thereof to a mixture comprising the test agent and the α-Adducin polypeptide or fragment thereof. In some embodiments, the test reaction mixture is formed by adding the α-Adducin polypeptide or fragment thereof to a mixture comprising the test agent and the α2-Na/K ATPase polypeptide or fragment thereof.

In some embodiments, the α-Adducin polypeptide or fragment thereof and/or the α2-Na/K ATPase polypeptide or fragment thereof is anchored to a solid support in the test reaction mixture. In some embodiments, the test reaction mixture is incubated under conditions conducive to the binding of the α2-Na/K ATPase polypeptide or fragment thereof to the α-Adducin polypeptide or fragment thereof. In some embodiments, the α2-Na/K ATPase polypeptide or fragment thereof and/or the α-Adducin polypeptide or fragment thereof is linked to a detectable moiety.

In some embodiments, the method further includes the step of isolating α2-Na/K ATPase polypeptide or fragment thereof bound to the α-Adducin polypeptide or fragment thereof from the α2-Na/K ATPase polypeptide or fragment thereof not bound to the α-Adducin polypeptide or fragment thereof. In some embodiments, the amount of complex in the test reaction mixture is determined by detecting the amount of α-Adducin polypeptide or fragment thereof bound to the α2-Na/K ATPase polypeptide or fragment thereof.

In some embodiments, the method further includes the step of isolating α-Adducin polypeptide or fragment thereof bound to the α2-Na/K ATPase polypeptide or fragment thereof from the α-Adducin polypeptide or fragment thereof not bound to the α2-Na/K ATPase polypeptide or fragment thereof. In certain embodiments, the amount of complex in the test reaction mixture is determined by detecting the amount of α-Adducin polypeptide or fragment thereof bound to the α2-Na/K ATPase polypeptide or fragment thereof.

In certain aspects, provided herein is a method of determining whether a subject has or is predisposed towards a neurodegenerative disease. In some embodiments, the method includes analyzing a cerebral spinal fluid sample from the subject to determine the expression level of α-Adducin and/or α2-Na/K ATPase in the sample, wherein elevated expression of α-Adducin and/or α2-Na/K ATPase (e.g., elevated expression compared to a control sample) indicates that the subject has or is predisposed towards a neurodegenerative disease. In some embodiments, the neurodegenerative disease is amyotrophic lateral sclerosis (ALS). Huntington's disease or spinocerebellar ataxias. In some embodiments, the neurodegenerative disease is ALS. In some embodiments, the method further includes treating the subject for a neurodegenerative disease according to the methods described herein if the subject is identified as having or being predisposed towards a neurodegenerative disease.

In some embodiments, any method can be used to analyze the sample. In some embodiments, the analysis of the sample comprises performing a nucleic acid amplification process on the sample. In some embodiments, the analysis of the sample comprises contacting the sample with a nucleic acid probe that hybridizes to an α-Adducin and/or α2-Na/K ATPase mRNA sequence or complement thereof (e.g., a detectably labeled nucleic acid probe and/or a nucleic acid probe immobilized on a solid support). In some embodiments, the analysis of the sample comprises the step of contacting the sample with an anti-α-Adducin and/or an anti-α2-Na/K ATPase antibody or antigen binding fragment thereof.

### BRIEF DESCRIPTION OF THE DRAWINGS

FIG. 1 shows upregulation of α-Adducin in SOD1^{G93A} astrocytes mediates non-cell autonomous degeneration of motor neurons. (A) Lysates of spinal cord from symptomatic SOD1^{G93A} transgenic mice and control non-transgenic mice were subjected to immunoblotting using an antibody that recognizes phosphorylated events in cells upon exposure to oxidative stress. (B) Lysates of spinal cords from symptomatic SOD1^{G93A} and control mice were subjected to immunoprecipitation using the α-Adducin antibody followed by immunoblotting with the phospho-antibody. (C) Immunoblots from spinal cord lysates show an increase in α-Adducin and phosphorylated α-Adducin relative to the internal control proteins ERK and 14-3-3β in symptomatic SOD1^{G93A} mice as compared to control wild type littermates (120 days). (D) Immunoblots show α-Adducin and phosphorylated α-Adducin are predominately expressed in primary glial cultures enriched with the astrocyte marker glia fibrillary acidic protein (GFAP) relative to primary motor neuron cultures enriched with the neuron marker β-tubulin. HSP60 is used as an internal control (lower panel). (E) Immunohistochemistry of symptomatic SOD1^{G93A} spinal cord sections revealed phosphorylated Ser436-α-Adducin (phospho-α-Adducin) co-localized with the astrocyte protein GFAP; scale bar 50 μm. (F) Co-cultured astrocytes and motor neurons were subjected to immunocytochemistry with antibodies recognizing the motor neuron nuclear protein Islet1 and the dendrite protein MAP2 scale bar 50 μm. Wild type astrocytes transfected with the control U6 or α-Adducin RNAi plasmid had little or no effect on motor neuron morphology or survival (upper and lower left panels); quantified (G and H). Control U6 SOD1^{G93A} astrocytes induced non-cell autonomous motor neuron cell death and dendrite abnormalities (upper right panel); quantified (G and H). α-Adducin knockdown in SOD1^{G93A} astrocytes protected motor neurons against the non-cell autonomous cell death and dendrite abnormality (lower right panel); quantified (G and H).

FIG. 2 shows knockdown of α-Adducin in SOD1^{G93A} mice suppresses motor neuron degeneration in vivo. (A) Schematic of unilateral intraspinal cord delivery of lentivirus expressing short hairpin RNAs targeting α-Adducin and encoding GFP (LV-Addi SOD1^{G93A}) or the corresponding control U6 lentivirus into 90 day old mice. Spinal cords from SOD1^{G93A} mice injected intraspinally were subjected to immunohistochemistry at end stage. End stage was defined as a time point at which the animal was unable to upright itself within 30 s of placement on its side. Immunohistochemistry with GFP in sections of the spinal cord in SOD1^{G93A} mice lumbar revealed delivery of control virus (LV-U6; panel B) or α-Adducin RNAi virus (LV-Addi; panel D) into the ventral horn; scale bar 100 μm. (B and D) Alternating GFP positive sections were subjected to immunohistochemistry using the GFP antibody and the neurofilment-SMI32 antibody, a motor neuron marker, or Nissl stained (bottom panels) for quantification of surviving motor neurons within GFP-labeled injected ventral horn and contralateral non-injected ventral horn (n≧20 sections per animal); scale bar 50 μm. (B and C) Control LV-U6 SOD1^{G93A} mice (n=5) displayed equivalent degeneration of motor neurons within injected GFP-labeled ventral horn and non-injected contralateral ventral horn (left panels). (D and E) α-Adducin knockdown in SOD1^{G93A} mice (LV-Addi SOD1^{G93A}; n=5) increased motor neuron survival within GFP-labeled injected ventral horn as compared to non-injected contralateral ventral horn (right panels).

FIG. 3 shows Enrichment of the α2-Na/K ATPase/α-Adducin complex in SOD1^{G93A} astrocytes triggers motor neuron degeneration. (A) Immunoblots from spinal cord lysates show an increase in the protein levels of α2-Na/K ATPase relative to the internal control proteins ERK and 14-3-3β in symptomatic SOD1^{G93A} mice as compared to control wild type littermates (120 days). (B) Immunoblots show α2-Na/K ATPase is upregulated in SOD1^{G93A} astrocytes as compared to non-transgenic controls. Knockdown of α-Adducin in SOD1^{G93A} astrocytes attenuated α2-Na/K ATPase protein levels. Protein levels are relative to ERK and 14-3-3β. (C) Co-cultured astrocytes and motor neurons were subjected to immunocytochemistry with the motor neuron nuclear protein Islet1 (red) and the dendrite protein MAP2 (green); scale bar 50 μm. Wild type astrocytes transfected with the control U6 or α2-Na/K ATPase RNAi plasmid had little or no effect on motor neuron morphology or survival (upper and lower left panels); quantification (D and E). Control U6 SOD1^{G93A} astrocytes induced non-cell autonomous motor neuron cell death and dendrite abnormalities (upper right panel); quantified (D and E). α2-Na/K ATPase knockdown in SOD1^{G93A} astrocytes protected motor neurons against the non-cell autonomous cell death and dendrite abnormalities (lower right panel); quantified (D and E). (F) Alternating GFP positive sections from SOD1^{G93A} mice injected intraspinally with lentivirus expressing α2-Na/K ATPase RNAi or control U6 were subjected to immunohistochemistry using the GFP and neurofilment-SMI32 antibodies or Nissl stained (bottom panels) for quantification of surviving motor neurons within GFP-labeled injected ventral horn and contralateral non-injected ventral horn at end stage (n≧20 sections per animal); scale bar 50 μm. End stage was defined as a time point at which the animal was unable to upright itself within 30 s of placement on its side. α2-Na/K ATPase knockdown in SOD1^{G93A} mice (LV-ATPi SOD1^{G93A}; n=5) increased motor neuron survival within GFP-labeled injected ventral horn as compared to non-injected contralateral ventral horn (lower panels). Arrowheads indicate surviving motor neurons; quantified (G). All data in bar charts show mean±s.e.m. Student t test for paired samples; *P<0.05, ***P<0.0001.

FIG. 4 shows heterozygous disruption of the α2-Na/K ATPase gene in SOD1^{G93A} mice suppresses motor neuron degeneration and enhances mouse lifespan. (A) Downregulation of α2-Na/K ATPase in SOD1^{G93A} astrocytes by crossing SOD1^{G93A} mice for α2-Na/K ATPase heterozygous null (right panel) protected motor neurons from non-cell autonomous cell death and dendrite abnormalities induced by control SOD1^{G93A} astrocytes (left panel); quantified (B and C); scale bar 50 μm. (D) Disease onset, i.e. initial day of weight loss, was significantly delayed in α2-Na/K ATPase heterozygous-null; SOD1^{G93A} mice (ATPase^{+/-}; n=11) as compared to control SOD1^{G93A} littermates (ATPase^{+/+}; n=11). (E) Early disease process, i.e. age at which 10% of weight loss is reached, was significantly delayed in α2-Na/K ATPase heterozygous-null; SOD1^{G93A} mice (ATPase^{+/-}; SOD1^{G93A}, red circles; n=11) as compared to control SOD1^{G93A} littermates (ATPase^{+/+}; SOD1^{G93A}; black squares; n=11); P=0.0042. (F) Early phase disease progression, i.e. days from onset to 10% weight loss, displayed no change between α2-Na/K ATPase heterozygous-null; SOD1^{G93A} mice and control SOD1^{G93A} littermates. (G) Late phase disease progression, i.e. from 10% weight loss to end stage, displayed a significant delay in α2-Na/K ATPase heterozygous-null SOD1^{G93A} mice (ATPase^{+/-}; n=11) as compared to control SOD1^{G93A} littermates (ATPase^{+/+}; n=11). (H) Kaplan-Meier survival plots show a substantial and significant increase in lifespan for α2-Na/K ATPase heterozygous-null; SOD1^{G93A} mice (ATPase^{+/-}; SOD1^{G93A}, circles; n=11) as compared to control SOD1^{G93A} littermates (ATPase^{+/+}; SOD1^{G93A}; black squares; n=11); P=0.0001. (I) Nissl stained sections from end stage control SOD1^{G93A} mice (n=5) and aged-matched SOD1^{G93A} littermates heterozygous-null for the α2-Na/K ATPase allele (n=5) display more than twice the number of motor neurons in ATPase^{+/-}; SOD1^{G93A} than control SOD1^{G93A} mice. Arrow heads indicate surviving motor neurons; quantification shown in (J); scale bar 50 μm.

FIG. 5 shows Na/K ATPase activity triggers degeneration of motor neurons. (A) Pharmacological inhibition of Na/K ATPase in control co-cultures non-transgenic astrocytes and motor neurons with ouabain (top second panel) and digoxin (top third panel) did not alter motor neuron survival or morphology; quantified (B and C). Control SOD1^{G93A} astrocytes (bottom first panel) induced non-cell autonomous motor neuron cell death and dendrite abnormalities; quantified (B and C). Pharmacological inhibition of Na/K ATPase with ouabain (bottom second panel) and digoxin (bottom third panel) was neuroprotective from the non-cell autonomous cell death and dendrite abnormalities induced by SOD1^{G93A} astrocytes (first panel); quantified (B and C); scale bar 50 μm.

FIG. 6 shows the α2-Na/K ATPase/α-Adducin complex is upregulated in spinal cord in familial and sporadic ALS patients. (A) Immunoblots from patients with familial ALS (n=5) show elevated protein levels of α2-Na/K ATPase (top panel) and α-Adducin (bottom panel) in spinal cord lysates as compared to control patients (n=3). ERK serves as loading control. Quantification of the relative densitometry protein levels for α2-Na/K ATPase and α-Adducin relative to the internal control ERK (B and C). (D) Immunoblots from patients with sporadic ALS (n=5) show elevated protein levels of α2-Na/K ATPase (top panel) and α-Adducin (bottom panel) in spinal cord lysates as compared to control patients (n=3). Quantification of the relative densitometry protein levels for α2-Na/K ATPase and α-Adducin relative to the internal control ERK (E and F). All data in bar charts show mean±s.e.m. Student t test for paired samples; *P<0.05.

FIG. 7 shows that the treatment of SOD1^{G93A} mice with intraperitoneally delivered digoxin compared to vehicle significantly increases the number of motor neurons identified in the ventral horn of lumbar spinal cord. Scale bar=50 μm. *** denotes P<0.0001, t-test, n=45 lumbar sections from n=7 mice per condition.

### DETAILED DESCRIPTION

### General

Described herein are methods of reducing neurodegeneration and/or treating or preventing a neurodegenerative disease or a neurodevelopmental disorder in a subject. For example, in some embodiments, the neurodegenerative disease is ALS, Huntington's disease, Alzheimer's disease, Parkinson's disease, spinal muscular atrophy (SMA), primary lateral sclerosis (PLS), progressive muscular atrophy (PMA), traumatic brain injury, spinocerebellar ataxias and/or progressive bulbar palsy (PBP). In some embodiments, the neurodevelopmental disorder is fragile X syndrome, Down's syndrome, Rett syndrome, intellectual disability, autism or an autism spectrum disorders such as Asperger syndrome. Also described herein are methods for the identification of agents useful in the foregoing methods.

In certain embodiments, provided herein are methods of treating and/or preventing a neurodegenerative disease or a neurodevelopmental disorder through the inhibition of the α2-Na/K ATPase/α-Adducin complex. In some embodiments, the method includes the step of inhibiting α2-Na/K ATPase. In some embodiments, the method includes the step of inhibiting α-Adducin. In some embodiments, the method includes the step of inhibiting a complex between α2-Na/K ATPase and α-Adducin.

Astrocytes play a fundamental role in the progression of diverse neurodegenerative diseases and neurodevelopmental disorders. As described in the Examples below, a complex composed of the ion pump α2-Na/K ATPase and the protein α-Adducin in SOD1^{G93A} astrocytes triggers the non-cell autonomous degeneration of motor neurons. Knockdown of α2-Na/K ATPase or α-Adducin in SOD1^{G93A} astrocytes profoundly inhibits their ability to induce degeneration in co-cultured primary motor neurons. In addition, in vivo knockdown of the α2-Na/K ATPase/α-Adducin complex by lentiviral-mediated RNAi in the spinal cord of SOD1^{G93A} mice protects motor neurons from degeneration in vivo. Inactivating one allele of the α2-Na/K ATPase gene in SOD1^{G93A} mice suppresses motor neuron degeneration and substantially increases healthspan and mouse lifespan. The Na/K ATPase small molecule inhibitors ouabain and digoxin block SOD1^{G93A} astrocyte-induced degeneration of co-cultured primary motor neurons. Finally, the α2-Na/K. ATPase/α-Adducin complex is substantially upregulated in the spinal cord in familial ALS bearing distinct SOD1 mutations as well as in sporadic ALS. These findings indicate that the α2-Na/K ATPase/α-Adducin complex plays a critical role in the pathogenesis of non-cell autonomous neurodegeneration and provides a drugable target in the treatment of neurodegenerative diseases.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” refers to both an intact antibody (i.e., a full length immunoglobulin molecule) and antigen binding antibody fragments. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen binding fragment” and “antigen-binding portion” of an antibody, as used herein, refers to antigen binding molecules that include one or more fragments of an antibody. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which an antibody is capable of binding. The term “extracellular epitope” refers to an epitope that is located on the outside of a cell's plasma membrane.

As used herein, the term “humanized antibody” refers to an antibody that has at least one CDR derived from a mammal other than a human, and a FR region and the constant region of a human antibody. A humanized antibody is useful as an effective component in a therapeutic agent according methods disclosed herein since antigenicity of the humanized antibody in human body is lowered.

As used herein, the terms “interfering nucleic acid,” “Inhibiting nucleic acid” are used interchangeably. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules. Such an interfering nucleic acids can be designed to block or inhibit translation of mRNA or to inhibit natural pre-mRNA splice processing, or induce degradation of targeted mRNAs, and may be said to be “directed to” or “targeted against” a target sequence with which it hybridizes. Inhibiting nucleic acids may include, for example, peptide nucleic acids (PNAs), locked nucleic acids (LNAs), 2′-O-Methyl oligonucleotides and RNA interference agents (siRNA agents). RNAi molecules generally act by forming a herteroduplex with the target molecule, which is selectively degraded or “knocked down,” hence inactivating the target RNA. Under some conditions, an interfering RNA molecule can also inactivate a target transcript by repressing transcript translation and/or inhibiting transcription of the transcript. An interfering nucleic acid is more generally said to be “targeted against” a biologically relevant target, such as a protein, when it is targeted against the nucleic acid of the target in the manner described above.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “sample” refers to a collection of cells or cell components (e.g., proteins, DNA, RNA) obtained from a subject. The sample may also contain compounds that are not naturally intermixed with the tissue in nature such as preservatives, anticoagulants, buffers, fixatives, nutrients, antibiotics or the like.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays described herein.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000) fold less than its affinity for binding to a non-specific and unrelated antigen/binding partner (e.g., BSA, casein).

An oligonucleotide “specifically hybridizes” to a target polynucleotide if the oligomer hybridizes to the target under physiological conditions, with a Tm substantially greater than 45° C., or at least 50° C., or at least 60° C.−80° C. or higher. Such hybridization corresponds to stringent hybridization conditions. At a given ionic strength and pH, the Tm is the temperature at which 50% of a target sequence hybridizes to a complementary polynucleotide. Again, such hybridization may occur with “near” or “substantial” complementarity of the antisense oligomer to the target sequence, as well as with exact complementarity.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Target Proteins

In certain embodiments, provided herein are methods of treating a neurodegenerative disease or a neurodevelopmental disorder by administering an agent that inhibits α2-Na/K ATPase and/or α-Adducin (referred to herein as “target proteins”). Inhibition of the target proteins can, for example, be via inhibition of protein activity or protein amount. For example, agents that inhibit the target protein include agents that reduce target protein activity, agents that increase target protein degradation, agents that inhibit transcription and/or translation of nucleic acids encoding the target protein and agents that increase degradation of nucleic acids encoding the target protein.

The Na/K ATPases are enzymes that catalyze the hydrolysis of ATP coupled with the exchange of sodium and potassium across the plasma membrane. Na/K ATPases are composed of two subunits, a catalytic α subunit and a non-catalytic β subunit. The four catalytic isoforms, α1-α4, display a unique tissue expression pattern. The α2 catalytic subunit is expressed in astrocytes and the central nervous system. The term α2-Na/K ATPase refers to a Na/K ATPase that includes an α2 catalytic subunit. The α2 subunit of the Na/K. ATPase is encoded by the ATP1A2 gene. The amino acid sequence of the human α2 subunit of the Na/K ATPase is available at NCBI accession number NP_000693.1 and is incorporated by reference herein. The nucleic acid sequence of the human α2 subunit of the Na/K ATPase mRNA is available at NCBI accession number NM_000702.3 and is incorporated by reference herein. The amino acid sequence of the human β1 subunit of the Na/K ATPase is available at NCBI accession number NP_001668.1 and is incorporated by reference herein. The nucleic acid sequence of the human β1 subunit of the Na/K ATPase mRNA is available at NCBI accession number NM_001677.3 and is incorporated by reference herein. The amino acid sequence of the human β2 subunit of the Na/K ATPase is available at NCBI accession number NP_001669.3 and is incorporated by reference herein. The nucleic acid sequence of the human β2 subunit of the Na/K ATPase mRNA is available at NCBI accession number NM_001678.3 and is incorporated by reference herein.

The α-Adducin is a membrane-cytoskeleton-associated protein that forms dimerizes with either β-Adducin or γ-Adducin to form Adducin. α-Adducin is encoded by the ADD1 gene. The amino acid sequence of the human α-Adducin is available at NCBI accession number NP_001110.2, NP_054908.2, NP_054909.2 and NP_789771.1, each of which is incorporated by reference herein. The nucleic acid sequence of the human α-Adducin mRNA is available at NCBI accession number NM_001119.4, NM_0141189.3, NM_0140190.3 and NM_176801.2, each of which is incorporated by reference herein.

### Small Molecule Agents

Certain embodiments of disclosed herein relate to methods of treating neurodegenerative diseases, such as ALS, or neurodevelopmental disorders. These methods include administering an agent that inhibits α2-Na/K ATPase or α-Adducin. Such agents include those disclosed below, those known in the art and those identified using the screening assays described herein.

In some embodiments, any agent that inhibits α2-Na/K ATPase or α-Adducin can be used to practice the methods disclosed herein. In some embodiments, the agent is a small molecule. For example, in some embodiments the agent is a cardiac glycoside. In some embodiments, the agent is digoxin, ouabain, digitoxin, proscillaridin A, digoxigenin, gitoxin, gitoxigenin, oleandrin, butalin, cinobufagenin, UNBS1450 or lanatoside C.

In some embodiments, the agent is digoxin or an active derivative thereof. Digoxin has the following chemical structure:

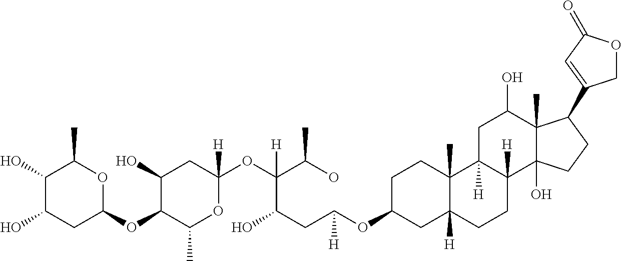

In some embodiments, the agent is ouabain or a derivative thereof. Ouabain has the following chemical structure:

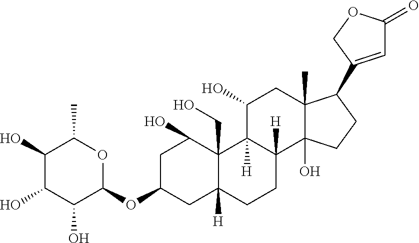

In some embodiments, the agent is digitoxin or a thereof. Digitoxin has the following chemical structure:

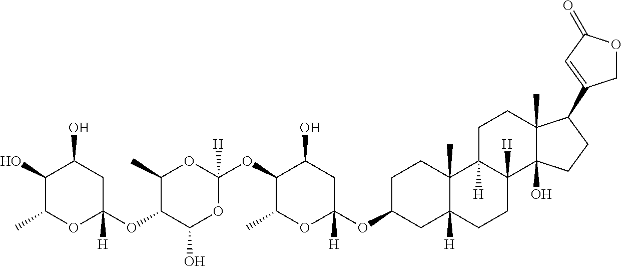

In some embodiments, the agent is proscillaridin A or a derivative thereof. Proscillaridin A has the following chemical structure:

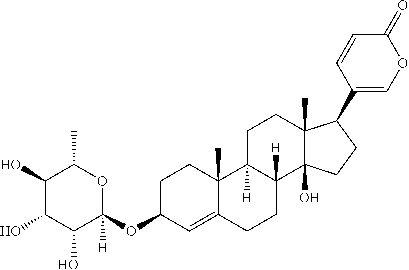

In some embodiments, the agent is digoxigenin or a derivative thereof. Digoxigenin has the following chemical structure:

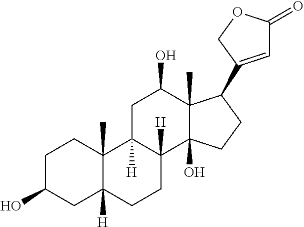

In some embodiments, the agent is gitoxin or a derivative thereof. Gitoxin has the following chemical structure:

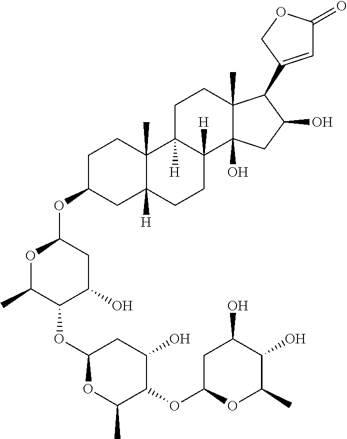

In some embodiments, the agent is gitoxigenin or a derivative thereof. Gitoxigenin has the following chemical structure:

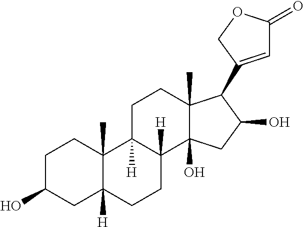

In some embodiments, the agent is oleandrin or a derivative thereof. Oleandrin has the following chemical structure:

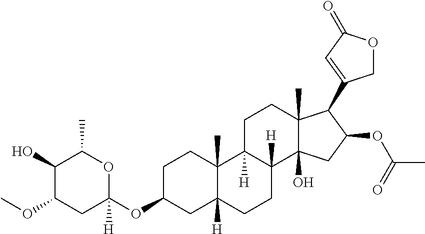

In some embodiments, the agent is butalin or a derivative thereof. Butalin has the following chemical structure:

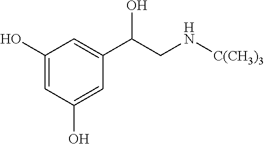 [SMILES: CC(C)(C)NCC(O)c1cc(O)cc(O)c1]

In some embodiments, the agent is cinobufagenin or a derivative thereof. Cinobufagenin has the following chemical structure:

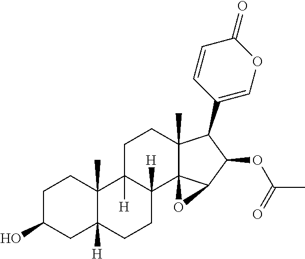

In some embodiments, the agent is UNBS1450 or a derivative thereof. UNBS1450 has the following chemical structure:

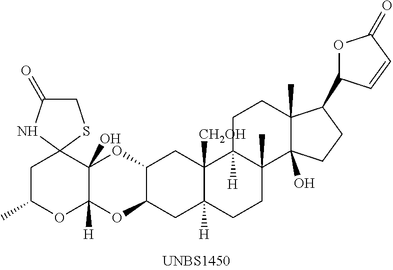

in some embodiments, the agent is lanatoside C or a derivative thereof. Lanatoside C has the following chemical structure:

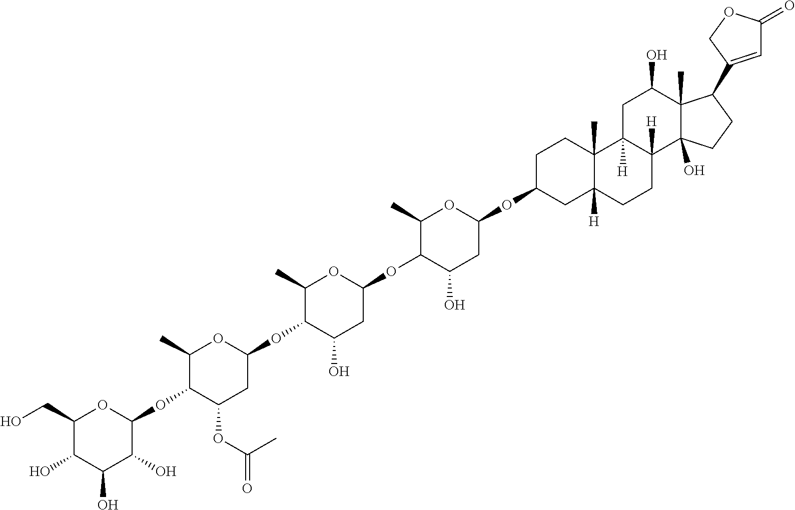

In some embodiments, assays used to identify agents useful in the methods described herein include a reaction between an α2-Na/K ATPase polypeptide or a fragment thereof and/or an α-Adducin polypeptide or a fragment thereof and a test compound. Agents identified via such assays, may be useful, for example, for treating or preventing neurodegenerative diseases or neurodevelopmental disorders.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994)J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods disclosed herein may be identified, for example, using assays for screening candidate or test compounds which inhibit complex formation between α2-Na/K ATPase and α-Adducin.

The basic principle of the assay systems used to identify compounds that inhibit complex formation between α2-Na/K ATPase and α-Adducin involves preparing a reaction mixture containing a α2-Na/K ATPase protein or fragment thereof and a α-Adducin protein or fragment thereof under conditions and for a time sufficient to allow the α2-Na/K ATPase protein or fragment thereof to form a complex with the α-Adducin protein or fragment thereof. In order to test an agent for modulatory activity, the reaction mixture is prepared in the presence and absence of the test compound. The test compound can be initially included in the reaction mixture, or can be added at a time subsequent to the addition of the α-Adducin protein or fragment thereof and the α2-Na/K ATPase protein or fragment thereof. Control reaction mixtures are incubated without the test compound or with a placebo. The formation of any complexes between the α-Adducin protein or fragment thereof and the α2-Na/K ATPase protein or fragment thereof is then detected. The formation of a complex in the control reaction, but less or no such formation in the reaction mixture containing the test compound, indicates that the compound interferes with the interaction of the α-Adducin protein or fragment thereof and the α2-No/K ATPase protein or fragment thereof.

The assay for compounds that modulate the interaction of the α-Adducin protein or fragment thereof and the α2-Na/K ATPase protein or fragment thereof may be conducted in a heterogeneous or homogeneous format. Heterogeneous assays involve anchoring either the α-Adducin protein or fragment thereof or the α2-Na/K ATPase protein or fragment thereof onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the α-Adducin protein or fragment thereof and the α2-Na/K ATPase protein or fragment thereof (e.g., by competition) can be identified by conducting the reaction in the presence of the test substance, i.e., by adding the test substance to the reaction mixture prior to or simultaneously with the α-Adducin protein or fragment thereof and the α2-Na/K ATPase protein or fragment thereof. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the α-Adducin protein or fragment thereof or the α2-Na/K ATPase protein or fragment thereof is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the α-Adducin protein or fragment thereof or the α2-Na/K ATPase protein or fragment thereof and drying. Alternatively, an immobilized antibody specific for the assay component to be anchored can be used for this purpose.

In related assays, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed the α-Adducin protein or fragment thereof or the α2-Na/K ATPase protein or fragment thereof, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above.

A homogeneous assay may also be used to identify inhibitors of complex formation. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the α-Adducin protein or fragment thereof and the α2-Na/K ATPase protein or fragment thereof.

### Interfering Nucleic Acid Agents

In certain embodiments, interfering nucleic acid molecules that selectively target α2-Na/K ATPase or α-Adducin are provided herein and/or used in methods described herein. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided hereion may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonuclotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAs cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40), 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002. The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNA, technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e 109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930, 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980) Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonzation-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### Antibody Agents

In certain embodiments, an antibody agent is used in the methods disclosed herein. In certain embodiments, the antibody agent binds to α2-Na/K ATPase. In some embodiments, the antibody agent binds to an extracellular domain of α2-Na/K ATPase.

Polyclonal antibodies can be prepared by immunizing a suitable subject (e.g. a mouse) with a polypeptide immunogen (e.g., a polypeptide having an amino acid sequence of α2-Na/K ATPase or a fragment thereof). The polypeptide antibody titer in the immunized subject can be monitored over time by standard techniques, such as with an enzyme linked immunosorbent assay (ELISA) using immobilized polypeptide. If desired the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for α2-Na/K ATPase can be identified and isolated by screening a recombinant combinatorial immunoglobulin library (e.g., an antibody phage display library or an antibody yeast display library) with the appropriate polypeptide (e.g. a polypeptide having an amino acid sequence of α2-Na/K ATPase or a fragment thereof) to thereby isolate immunoglobulin library members that bind the polypeptide.

Additionally, recombinant antibodies specific for α2-Na/K ATPase, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. No. 4,816,567; U.S. Pat. No. 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sc. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for α2-Na/K ATPase can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies described herein are able to bind to an extracellular epitope of α2-Na/K ATPase with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis. In some embodiments, the binding of the antibody to α2-Na/K ATPase substantially inhibits the ability of α2-Na/K ATPase to catalyze the hydrolysis of ATP or exchange sodium and potassium across the plasma membrane.

### Polypeptide Agents

In certain embodiments, a polypeptide agent is used in the methods disclosed herein.

In some embodiments, the polypeptide agent is an isolated polypeptide comprising a α-Adducin domain or fraction thereof required for α2-Na/K ATPase to form a complex with α-Adducin In some embodiments, the polypeptide agent is an isolated polypeptide comprising a α2-Na/K ATPase domain or fraction thereof required for α-Adducin to form a complex with α2-Na/K ATPase. Such polypeptides can be useful, for example, for inhibiting the ability of α2-Na/K ATPase to form a complex with α-Adducin. In some embodiments, the polypeptide comprises at least 5, 6, 7, 8, 9, 10, 11, 12, 13, 14, 15, 16, 17, 18, 19, 20, 25, 30, 35, 40, 45, 50, 60, 70, 80, 90 or 100 consecutive amino acids of an amino acid sequence α-Adducin protein. In some embodiments, the polypeptide comprises at least 5, 6, 7, 8, 9, 10, 1, 12, 13, 14, 15, 16, 17, 18, 19, 20, 25, 30, 35, 40, 45, 50, 60, 70, 80, 90 or 100 consecutive amino acids of an amino acid sequence α2-Na/K ATPase protein.

In some embodiments, the polypeptides disclosed herein can be isolated from cells or tissue sources by an appropriate purification scheme using standard protein purification techniques. In another embodiment, polypeptides are produced by recombinant DNA techniques. Alternatively, polypeptides disclosed herein can be chemically synthesized using standard peptide synthesis techniques.

The polypeptides described herein can be produced in prokaryotic or eukaryotic host cells by expression of polynucleotides encoding a polypeptide(s) described herein. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152. Guide to Molecular Cloning Techniques (1987), Academic Press, Inc., San Diego. Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

### Therapeutic Methods

Provided herein are methods for the treatment of neurodegenerative disease and/or the prevention of neurodegeneration. In some embodiments, the neurodegenarative disease is ALS, Huntington's disease, Alzheimer's disease, Parkinson's disease, SMA, PLS, PMA, traumatic brain injury, spinocerebellar ataxias or PBP. In some embodiments, the neurodegenerative disease is ALS. Also provided herein are methods of treating neurodevelopmental disorders (e.g., fragile X syndrome, Down's syndrome, Rett syndrome, intellectual disability, autism or an autism spectrum disorders such as Asperger syndrome) through the inhibition of α2-Na/K ATPase and/or α-Adducin.

The methods described herein can be used to treat any subject in need thereof. As used herein, a “subject in need thereof” includes any subject that has a neurodegenerative disease (e.g., ALS, Huntington's disease, Alzheimer's disease, Parkinson's disease, SMA, PLS, PMA, traumatic brain injury, spinocerebellar ataxias or PBP) or neurodevelopmental disorder, and well as any subject with an increased likelihood of acquiring a neurodegenerative disease or neurodevelopmental disorder. In certain embodiments, the subject in need thereof carries a gene mutation associated with a neurodegenerative disease, such as a mutated SOD1 gene. In some embodiments the subject in need thereof has at least one family member who has a neurodegenerative disease.

The pharmaceutical compositions disclosed herein may be delivered by any suitable route of administration, including orally and parenterally. In certain embodiments the pharmaceutical compositions are delivered generally (e.g. via oral or parenteral administration). In certain other embodiments the pharmaceutical compositions are delivered locally through direct injection into a specific tissue (e.g., central nervous system tissue and/or peripheral nervous system tissue).

The dosage of the subject agent may be determined by reference to the plasma and/or cerebrospinal fluid (CSF) concentrations of the agent. For example, the maximum plasma concentration (Cmax) and the area under the plasma concentration-time curve from time 0 to infinity (AUC(0-4)) may be used. Dosages include those that produce the above values for Cmax and AUC(0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

### EXEMPLIFICATION

### Experimental Procedures

### Human Tissue

All human spinal cord tissue samples were acquired by way of an Investigational Review Board and Health Insurance Portability and Accountability Act compliant process. The SOD1 A4V, V148G, and E100G nervous system were confirmed by DNA testing. All patients who had been followed during the clinical course of their illness had met E1 Escorial criteria for definite ALS. Upon death, autopsies were performed immediately by an on-call tissue acquisition team. Control spinal cord tissue samples were from patients from the hospital's critical care unit when life support was withdrawn or patients on hospice. Tissue samples were completed within 4-6 hours of death and the entire motor system was dissected and elaborately archived for downstream applications by creating two parallel tissue sets from alternating adjacent regions. For biochemical studies, segments were embedded in cutting media, frozen on blocks of dry ice, and stored at −80° C.

### Animals

Transgenic mice overexpressing mutant G93A human superoxide dismutase 1 (Jackson Laboratory stock#002726). α2-Na/K ATPase null heterozygous (provided by Dr. Jerry B. Lingrel) and C57BL6 (Charles River) were used. All analysis was done with littermate mice derived from SOD1^{G93A} heterozygous mating with non-transgenic wild type and heterozygous α2-Na/K ATPase mice. All animal experiments were conducted under the institutional guidelines and were approved by the Institutional Animal Care and Use Committee (IACUC).

### Plasmids

RNAi plasmids were designed as described in Gaudilliere et al., J. Biol. Chem. 277:46442-46446 (2002), which is hereby incorporated by reference. The U6/α-Adducin plasmids were cloned using the following primers: 5′-agt cga aga cta agt gga ctt-3′ and 5′-agt cga aga cta agt gga cta-3′. The U6/α2-Na % K ATPase plasmids were cloned using the following primers: 5′-gca tca tat cag agg gta acc-3′ and 5′-gtg gca aga aga aac aga aac-3′. α-Adducin and α2-Na/K ATPase were cloned from C57BL/6J mice cDNA and inserted into the pcDNA3 vector (Invitrogen) at the ECOR1 and Xho1 sites. The primer for site direct mutagenesis for RNAi-resistant form of α-Adducin is as followed 5′-aac gga age agt ccc aaA tcA aaA acA aaA tgg acA aaa gag gat gga cat ag-3.

### Antibodies

Antibodies to the following were used: α-Adducin (H-100, Santa Cruz), Phospho-Ser436-Adducin (Ser436, Santa Cruz) α2-Na/k ATPase (A-16, Santa Cruz), α2-Na/k ATPase (a5, Developmental Studies Hybridoma Bank), Islet-1 (39.4D5, Developmental Studies Hybridoma Bank), MAP2 (ab5622, Fisher), glial fibrillary acidic protein (AB5804, Millipore), Iba-1 (019-19741, WACO), 14-3-3β (H-8, Santa Cruz), ERK (Molecular Probes) Superoxide Dismutase (SD-G6, SIGMA), ERK (4685, Cell Signaling) and GFP (Molecular Probes).

### Protein Biochemistry

To determine the relative amounts of total proteins, whole spinal cords from aged match littermate mice were homogenized in 50 mM Tris-buffer (pH 7.2), 100 mM NaCl, 1 mM EDTA, 1 mM PMSF and a cocktail of protease and phosphatase inhibitors. Equal concentrations from each sample were solubilized in SDS-loading buffer and boiled for 10 min. For analyses of α2-Na/K ATPase protein levels, equal concentrations (1 mg/ml) from each sample were solubilized in SDS-loading buffer. Using an ultrasonic water sonicator protein samples in SDS-loading buffer were sonicated in an ice bath to prevent oligomerization of the α2-Na/K ATPase (6 pulses for 30 sec. with 30 sec. intervals). Protein quantification was carried out by immunoblotting using Amersham ECL plus and analyzed with Bio-Rad ChemiDoc gel imaging system. Signals were normalized for internal controls run on the same blots. For immunoprecipitation of α-Adducin, proteins were extracted under non-denaturing (1% Triton X-100 for 1 h at 4° C.) and denaturing conditions (1% SDS and 0.5% DOC for 1 h at 4° C.) and the insoluble material was removed by centrifugation (14,000 RPMs for 10 min.). Following the denaturing conditions the supernatant was diluted to a final concentration of 0.33% SDS and 0.16% Triton. The supernatants were precleared with protein A for 30 min. at 4′C prior to the addition of α-Adducin antibody for 1 hr at 4° C. For co-immunoprecipitation of α-Adducin and α2-Na/K ATPase, immunoprecipitated α-Adducin antibody was eluted from protein A with 0.2M Glycine pH 2.5 on ice for 15 min followed by quenching with 1 m Tris. pH 8.8.

### Primary Astrocyte Cultures

Monolayer astrocytes culture from P2 SOD1^{G93A} and non-transgenic littermate mice were prepared as described in Di Giorgio et al., Nat. Neurosci. 10:608-614 (2007) and Nagai et al., Nat. Neurosci. 10:615-622 (2007), each of which is incorporated by reference. Astrocytes were plated in DMEM (GIBCO) supplemented with 10% FBS containing 100 U/ml penicillin and 100 mg/ml streptomycin for two weeks or until confluent. Once confluent, cells were replated onto glass cover slips precoated with poly-D-lysine at a density of 40,000 cells/well in a 24-well dish. For RNAi experiment astrocyte cultures were transfected with RNAi or control U6 plasmid using Lipofectamine 2000 (Invitrogen) according to the guidelines of the manufacturer four days prior to co-culturing with motor neurons.

### Primary Motor Neuron Cultures and Morphological Analyses

Spinal motor neuronal cultures from E12.5 wild-type rodents were performed as described in Gingras et al., J. Neurosci. Methods 163:11-118 (2007), which is hereby incorporated by reference. Cultures were plated astrocyte monolayers at 12,000 cells/well in Neurobasal-A medium (GIBCO) supplemented with 2% B-27 serum free supplement and 0.5 mM glutamine; DMEM/F12 (3:1, v/v ratio) with a cocktail of trophic factors composed of 0.5 ng/ml glia-derived neurotrophic factor, 1 ng/ml brain-derived neurotrophic factor and 10 ng/ml ciliary neurotrophic factor (trophic factor cocktail, R&D Systems).

Motor neurons were fixed after 7 days of co-cultures with astrocytes and immunostained with Islet1, MAP2 and Hoechst 33258 and subjected to cell survival. Approximately 150 cells were counted per cover slip. For pharmacological inhibition of Na/K ATPase in co-cultures. 1.0 μM of ouabian or digoxin was added 4 hours following the co-culturing of motor neurons with control or G93A astrocytes (DIV 0). This was followed by administration of 0.5 μM of ouabain or digoxin every 48 hours (DIV 2, DIV 4, and DIV 6). For motor neuron morphology images were taken using a NIKON eclipse TE2000 epifluorescence microscope using a digital CCD camera (DIAGNOSTIC instruments) and imported into the SPOT imaging software. Approximately 40 cells were analyzed per cover slip.

### Morphological Studies

All morphological studies were performed as described in Chandra et al., Cell 123:383-396 (2005), which is incorporated by reference. Briefly, mice were deeply anesthetized and perfused with 4% paraformaldehyde in phosphate buffer. Spinal cords were post-fixed in the same fixative for 4 hours and processed for cryoprotective embedding. Immunohistochemistry analyses were carried out on 30 um cryosections from spinal cords with the indicated antibodies. For measurement of motor neurons survival alternating GFP positive sections were either immunostained or Nissl stained and images were taken using a NIKON eclipse TE2000 epifluorescence microscope or brightfield using a digital CCD camera (DIAGNOSTIC instruments) and imported into the SPOT imaging software. For lentiviral mediated RNAi a minimum of 20 sections were quantified for surviving motor neurons in the injected and non-injected ventral horns from Nissl stained and immunofluorescence labeled sections. For digoxin treated mice, alternating section for a total of 45 sections per spinal cord were quantified for motor neuron survival.

### Lentiviral Injections

Lentivirus was produced in 293T cells and concentrated by ultracentrifuge. Surgical procedures were performed as described in Raoul et al., Nat. Med. 11:423-428 (2005), which is hereby incorporated by reference. Briefly, 90 day old mice were anaesthetized with ketamine/xylazine intraperitoneal (90-200 mg/kg ketamine/10 mg/kg xylazine). A 2 cm longitudinal skin incision was made above the lumbar region under a dissecting microscope. Using a dental drill a small (1 mm) hole was made into the spinal cord. The lentiviral solution was injected into the L3-L4 region using a stereotaxic frame (Stoelting Co.) at 2 mm unilaterally. The viral solution (50 nl) was injected 25 times per animal with 45 seconds intervals using a fine micropipette (Drummond 30 ul microcapillaries pulled with P-9 capillary puller, Sutter Instruments) and a Nanoject II (Drummond). The micropipette was then left for an additional 5 min and gently withdrawn.

### Statistics

Statistical analyses were done using GraphPad Prism 4 software. Bar graphs are presented as the mean±SEM. For experiments in which only two groups were analyzed, the t-test was used. Comparisons within multiple groups were done by analysis of variance (ANOVA).

### Example 1

### Upregulation of α-Adducin in SOD1^{G93A} Astrocytes Induces Non-Cell Autonomous Degeneration of Motor Neurons

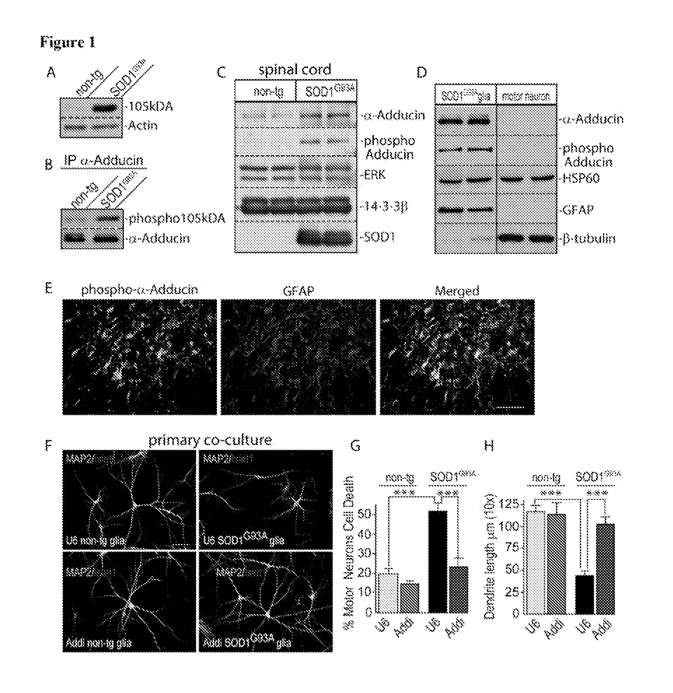

Using an antibody that recognizes phosphorylated events in cells upon exposure to oxidative stress, a 105 kDa immunoreactive protein band was identified that was enriched in lysates of spinal cord from symptomatic SOD1^{G93A} mice at 120 days of age as compared to age-matched wild type littermate mice (FIG. 1A). Upon treatment of symptomatic SOD1^{G93A} spinal cord lysates with λ-phosphatase the immunoreactive 105 kDa protein band was eliminated. Mass spectrometry analysis following immunoprecipitation assays led to the identification of α-Adducin as the putative phosphorylated protein in SOD1^{G93A} spinal cords. The mass spectrometry analysis was validated by immunoprecipitating α-Adducin and immunoblotting with the phospo-antibody, confirming the identity of α-Adducin in symptomatic SOD1^{G93A} mice (FIG. 1B). In other experiments, Ser436 was identified as the site of α-Adducin phosphorylation in lysates of SOD1^{G93A} spinal cords. Importantly, immunoblotting of α-Adducin in symptomatic and non-symptomatic SOD1^{G93A} spinal cord lysates revealed that α-Adducin is upregulated at disease onset, and upregulation of α-Adducin persists through the degenerative process in SOD1^{G93A} mice (FIG. 1C).

The cellular origin of α-Adducin in SOD1^{G93A} mice was next determined. In immunoblotting analysis of primary SOD1^{G93A} glial cells and motor neurons, α-Adducin and Ser436-phosphorylated α-Adducin were predominantly expressed in astrocytes rather than motor neurons (FIG. 1D). In complementary immunohistochemical analyses, Ser436-phosphorylated α-Adducin co-localized with the astrocyte marker glia fibrillary acidic protein (GFAP) in spinal cord of symptomatic SOD1^{G93A} mice (FIG. 1E). The localization and abundance of α-Adducin in astrocytes in symptomatic SOD1^{G93A} mice raised the question of whether α-Adducin might play a role in the toxic gain of function in SOD1^{G93A} astrocytes.

To characterize α-Adducin function in neurodegeneration, a cell culture model was employed in which SOD1^{G93A} astrocytes are co-cultured with primary spinal cord motor neurons, which recapitulates the non-cell autonomous degeneration of motor neurons in vivo. Using a plasmid-based method of RNA interference (RNAi), the efficient knockdown of α-Adducin in SOD1^{G93A} astrocytes was induced. SOD1^{G93A} astrocytes, but not wild type astrocytes, transfected with the control U6 RNAi plasmid induced cell death and substantial reduction in total dendrite length in motor neurons (FIG. 1F-1H), confirming that mutant SOD1 astrocytes trigger non-cell autonomous degeneration of motor neurons. Importantly, knockdown of α-Adducin in SOD1^{G93A} astrocytes protected motor neurons against the non-cell autonomous induction of motor neuron cell death (FIGS. 1F and 1G). Whereas control SOD1^{G93A} astrocytes induced cell death in 50% of co-cultured motor neurons, α-Adducin knockdown SOD1^{G93A} astrocytes induced cell death in only 23% of co-cultured motor neurons (FIGS. 1F and 1G). Likewise, knockdown of α-Adducin in SOD1^{G93A} astrocytes prevented the ability of SOD1^{G93A} astrocytes to induce abnormalities in motor neuron dendrite morphology (FIGS. 1F and 1H). In control analyses, knockdown of α-Adducin in non-transgenic astrocytes had little or no effect on the survival or morphology of co-cultured motor neurons (FIGS. 1F-1H).

To determine the specificity of the α-Adducin RNAi-induced neuroprotective phenotype in SOD1^{G93A} astrocytes, a rescue experiment was performed. An RNAi-resistant form of α-Adducin (Add-Res) was expressed in the background of α-Adducin RNAi in SOD1^{G93A} astrocytes. Expression of α-Adducin rescue (Add-Res) in SOD1^{G93A} astrocytes reversed the ability of α-Adducin RNAi to protect co-cultured motor neurons from cell death and impairment of dendrite morphology. These data indicate that the α-Adducin RNAi-induced neuroprotective effect is the result of specific knockdown of α-Adducin in SOD1^{G93A} astrocytes rather than off-target effects of RNAi. Together, the data suggest that α-Adducin in SOD1^{G93A} astrocytes plays a critical role in the non-cell autonomous degeneration of motor neurons.

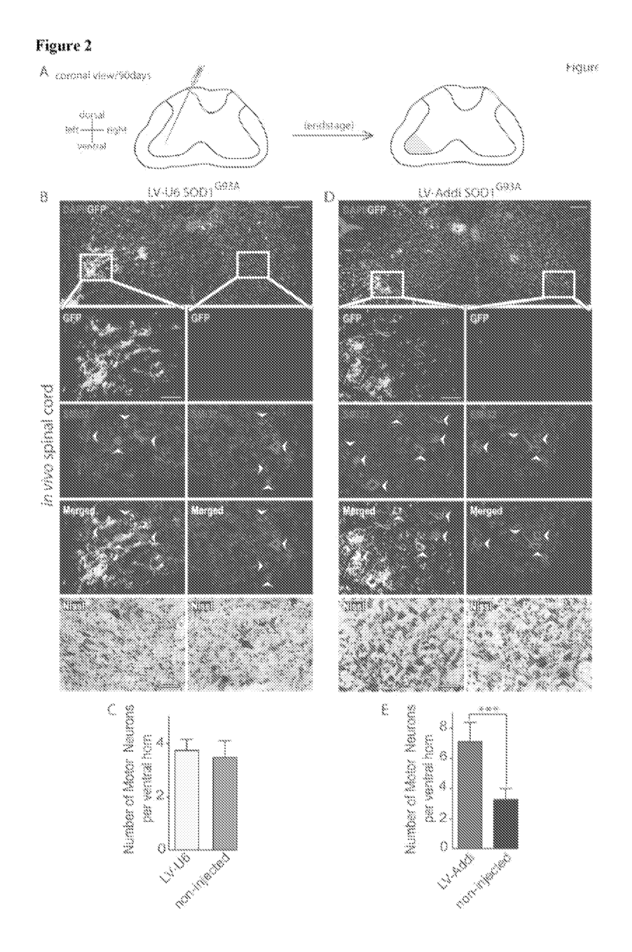

The impact of α-Adducin knockdown on neurodegeneration in the spinal cord of SOD1^{G93A} mice was assessed in vivo. Lentivirus encoding α-Adducin short hairpin RNAs and GFP (LV-Addi) or the corresponding control lentivirus (LV-U6) were injected unilaterally in the lumbar spinal cord in SOD1^{G93A} mice (FIG. 2A). This method of intraspinal RNAi allowed comparison of surviving motor neurons in the injected ventral horn with the non-injected contralateral ventral horn within the same spinal cord sections. Viruses were injected in SOD1^{G93A} mice at 90 days of age, when α-Adducin is upregulated and early gliosis has set in but prior to significant loss of motor neurons. Injection of control lentivirus in SOD1^{G93A} mice (LV-U6 SOD1^{G93A}) had little or no effect on the survival of ventral horn motor neurons in vive (FIG. 2B-2C). By contrast, α-Adducin knockdown in SOD1^{G93A} mice (LV-Addi SOD1^{G93A}) strongly suppressed motor neuron degeneration in vivo (FIG. 2D-2E). Whereas the α-Adducin knockdown mice harbored 7.08±1.27 motor neurons within the GFP-labeled ventral horn injected with α-Adducin RNAi virus, the contralateral non-injected ventral horn contained only 3.23±0.73 motor neurons (FIG. 2D-2E). In control analyses, it was confirmed that α-Adducin RNAi induced the knockdown of α-Adducin within the GFP-labeled injected ventral horn. In other control experiments, α-Adducin knockdown had little effect on gliosis or on the presence or migration of microglia. Together, these results indicate that α-Adducin plays a critical role in motor neuron degeneration in mutant SOD1^{G93A} mice in vivo.

### Example 2

### Enrichment of the α2-Na/K ATPase/α-Adducin Complex in SOD1^{G93A} Astrocytes Triggers Motor Neuron Degeneration

The mechanism underlying the novel function of α-Adducin in neurodegeneration was investigated. Immunoprecipitation of α-Adducin followed by mass spectrometry (IP-MS) in lysates of spinal cord from symptomatic SOD1^{G93A} mice was performed. These analyses revealed the ion pump α2-Na/K ATPase as an interactor of α-Adducin in symptomatic SOD1^{G93A} spinal cord lysates.

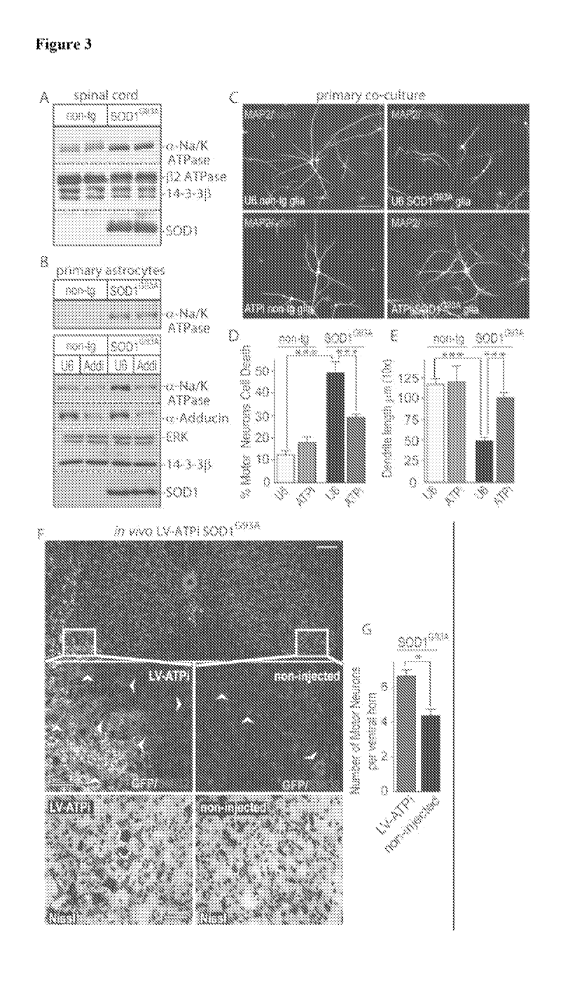

The interaction of μ-Adducin with α2-Na/K ATPase in symptomatic SOD1^{G93A} spinal cord lysates was validated using co-immunoprecipitation assays. Next, the expression of α2-Na/K ATPase in spinal cord of symptomatic SOD1^{G93A} mice was examined. Ass depicted in FIG. 3A, α2-Na/K ATPase was upregulated in symptomatic SOD1^{G93A} mice. The increase in α2-Na/K ATPase protein levels was also evident in primary SOD1^{G93A} astrocytes (FIG. 3B). The knockdown of α-Adducin in SOD1^{G93A} astrocytes reduced the levels of α2-Na/K ATPase in these cells (FIG. 3B). These data indicate that upregulation of α2-Na/K ATPase might act in concert with upregulated α-Adducin to trigger the toxic gain of function in SOD1^{G93A} astrocytes.

The role of α2-Na/K-ATPase in the toxic gain of function of SOD1^{G93A} astrocytes was next examined. Knockdown of α2-Na/K ATPase in non-transgenic control glia had little or no effect on motor neuron survival or dendrite morphology (FIGS. 3C-3E). In contrast, knockdown of α2-Na/K ATPase in SOD1^{G93A} astrocytes protected co-cultured primary motor neurons against non-cell autonomous cell death and impairment of dendrite morphology (FIGS. 3C-3E). These data indicate that knockdown of α2-Na/K ATPase phenocopies the neuroprotective effects of α-Adducin knockdown in SOD1^{G93A} astrocytes.

The role of α2-Na/K ATPase in SOD1^{G93A}-dependent neurodegeneration was next assessed in vive. A lentiviral approach was used to induce knockdown of α2-Na/K ATPase in the lumbar spinal cord in SOD1^{G93A} mice. Just as in the α-Adducin experiments in vivo, injection of control lentivirus in SOD1^{G93A} mice (LV-U6 SOD1^{G93A}) had no effect on motor neuron survival. By contrast, knockdown of α2-Na/K ATPase in SOD1^{G93A} mice by lentivirus (LV-ATPi SOD1^{G93A}) suppressed the degeneration of spinal cord motor neurons in vivo. Whereas the GFP-labeled injected ventral horn in the α2-Na/K ATPase knockdown mice harbored 6.7±0.36 motor neurons, only 4.38±0.40 motor neurons were present in the non-injected contralateral ventral horn in these mice (FIGS. 3F and 3G). Notably. α2-Na/K ATPase knockdown had little or no effect on the presence or abundance of astrocytes or microglia in the ventral horns of SOD1^{G93A} mice. These data show that α2-Na/K ATPase knockdown in SOD1^{G93A} mice suppresses motor neuron degeneration in vivo.

### Example 3

### Heterozygous Disruption of the α2-Na/K ATPase Gene in SOD1^{G93A} Mice Suppresses Motor Neuron Degeneration and Enhances Mouse Lifespan

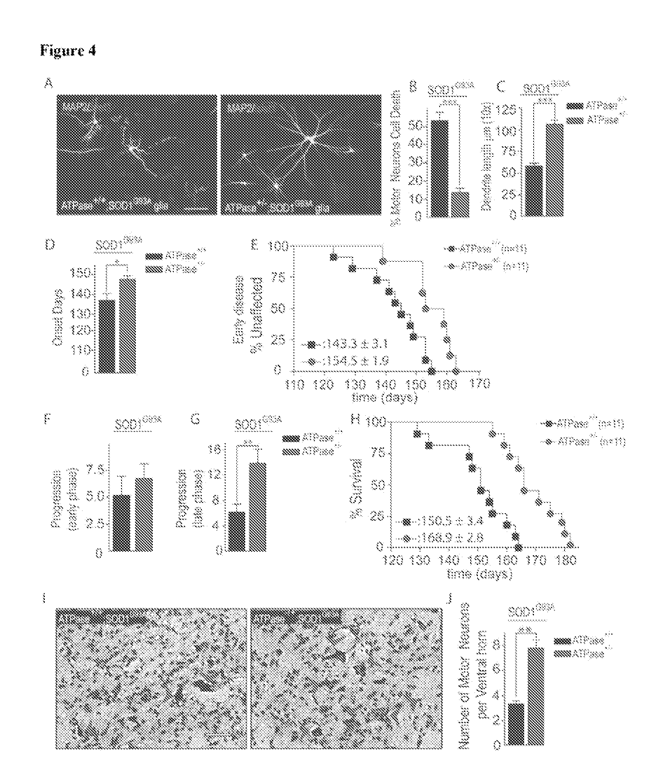

A genetic knockout approach was used to define the role of α2-Na/K ATPase in neurodegeneration in SOD1^{G93A} mice. Although complete absence of α2-Na/K ATPase leads to embryonic lethality, heterozygous-null mice expressing approximately 50% of α2-Na/K ATPase protein display no gross abnormalities. The ability of astrocytes from heterozygous-null α2-Na/K ATPase^{+/-}; SOD1^{G93A} mice (ATPase^{+/-}; SOD1^{G93A}) to induce cell death of co-cultured motor neurons was determined. Control SOD1^{G93A} astrocytes (ATPase^{+/+}; SOD1^{G93A}) induced non-cell autonomous cell death in 53% of co-cultured motor neurons (FIGS. 4A and 4B). In contrast, heterozygous-null α2-Na/K ATPase^{+/-}; SOD1^{G93A} astrocytes (ATPase^{+/-}; SOD1^{G93A}) induced non-cell autonomous cell death in only 14% of motor neurons (FIGS. 4A and 4B). Likewise, ATPase^{+/-}; SOD1^{G93A} astrocytes failed to induce dendrite abnormalities in motor neurons as compared to control SOD1^{G93A} astrocytes (FIGS. 4A and 4C). These data corroborate the results of knockdown analyses and buttress the conclusion that α2-Na/K ATPase plays a critical role in non-cell autonomous degeneration of motor neurons.

The genetic knockout approach facilitated analysis of the role of α2-Na/K ATPase^{+/-} in the motor neuron disease phenotype of mutant SOD1 mice. Disease onset, progression, and lethality was evaluated in heterozygous-null α2-Na/K ATPase; SOD1^{G93A} mice and control SOD1^{G93A} littermates. Disease onset, defined as first day of weight loss, was significantly delayed in ATPase^{+/-}; SOD1^{G93A} mice (ATPase^{+/-}) as compared to control ATPase^{+/+}; SOD1^{G93A} mice (ATPase^{+/+}) (FIG. 4D). Accordingly, disruption of the α2-Na/K ATPase gene in SOD1^{G93A} mice delayed the age at which early disease 10% weight loss was reached a measurement of early disease (FIG. 4E). Early phase disease progression, as measured from the first day of weight loss to 10% weight loss, was not significantly altered between ATPase^{+/-}; SOD1^{G93A} and control SOD1^{G93A} littermates (FIG. 4F). In contrast, late phase disease progression, as measured from 10% weight loss to end stage, was significantly delayed in ATPase^{+/-}; SOD1^{G93A} mice compared to control SOD1^{G93A} littermates (FIG. 4G). Strikingly, the overall survival of SOD1^{G93A} mice was increased upon reducing the expression of α2-Na/ATPase to an average life span of 168.9±2.75 compared to 150.5±3.3 days in control SOD1^{G93A} mice (FIG. 3H). The ATPase^{+/-}; SOD1^{G93A} mice were substantially more mobile and heather at the time that the control SOD1^{G93A} mice were at end stage of the disease (Movie S1. and Movie S2.). Thus reducing the expression of α2-Na/K ATPase in SOD1^{G93A} mice delays the onset and slows the progressive process of neurodegeneration, thereby substantially increasing healthspan and lifespan.

To determine if the improvement in mortality and morbidity of SOD1^{G93A} mice that are heterozygous for the α2-Na/K ATPase in SOD1^{G93A} gene is associated with suppression of motor neuron degeneration in vivo, motor neurons were quantified at end stage in control SOD1^{G93A} mice and aged-matched SOD1^{G93A} littermates heterozygous-null for the α2-Na/K ATPase allele. Control SOD1^{G93A} mice harbored 3.31±0.17 motor neurons per ventral horn (FIGS. 4I and 4J). By contrast, littermate heterozygous-null α2-Na/K ATPase; SOD1^{G93A} mice had more than twice the number of motor neurons at 7.81±0.41 per ventral horn (FIGS. 4I and 4J). These results demonstrate that reducing the expression of α2-Na/K ATPase in SOD1^{G93A} mice delays motor neuron degeneration. Collectively, the data suggest that α2-Na/K ATPase plays a critical role in neurodegeneration.

### Example 4

### Na/K ATPase Activity Triggers Degeneration of Motor Neurons

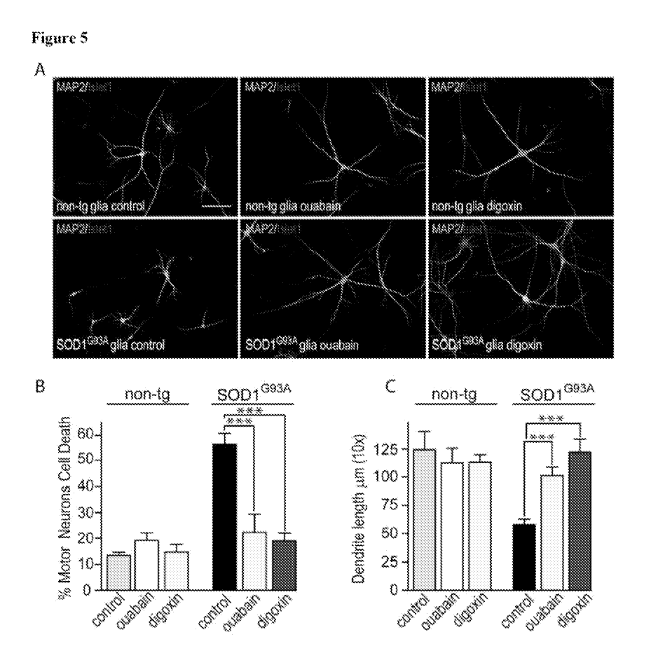

The elevated levels of the α2-Na/K ATPase/α-Adducin complex in SOD1^{G93A} astrocytes raised the question of whether the activity of α2-Na/K ATPase per se plays a pathogenic role in the toxic gain of function of SOD1^{G93A} astrocytes. The Na/K ATPase small molecule inhibitors ouabain and digoxin were used, the latter used widely as a therapeutic drug in treatment of heart failure, to assess the role of Na/K ATPase activity in the toxic effects of SOD1^{G93A} astrocytes. Following the co-culturing of motor neurons with control and mutant SOD1^{G93A} astrocytes, ouabain, digoxin or control vehicle were added at a final concentration of 1 μm, which is sufficient to inhibit the α2-Na/K ATPase in primary glia cells. Inhibition of Na/K ATPase with ouabain or digoxin substantially reduced motor neuron cell death induced by SOD1^{G93A} astrocytes to 22% and 19% respectively as compared to 56% motor neuron cell death in cultures treated with vehicle (FIGS. 5A and 5B). Likewise, both ouabain and digoxin prevented impairment of dendrite morphology in motor neurons induced by SOD1^{G93A} astrocytes (FIGS. 5A and 5C). Control analyses, exposer of co-cultures of motor neurons and control astrocytes to ouabain and digoxin did not alter survival or morphology of motor neurons (FIG. 5A-5C). These results support the conclusion that the catalytic activity of α2-Na/K ATPase/α-Adducin complex in SOD1^{G93A} astrocytes triggers non-cell autonomous degeneration of motor neurons.

### Example 5

### The α2-Na/K ATPase/α-Adducin Complex is Upregulated in Spinal Cord in Individuals with ALS

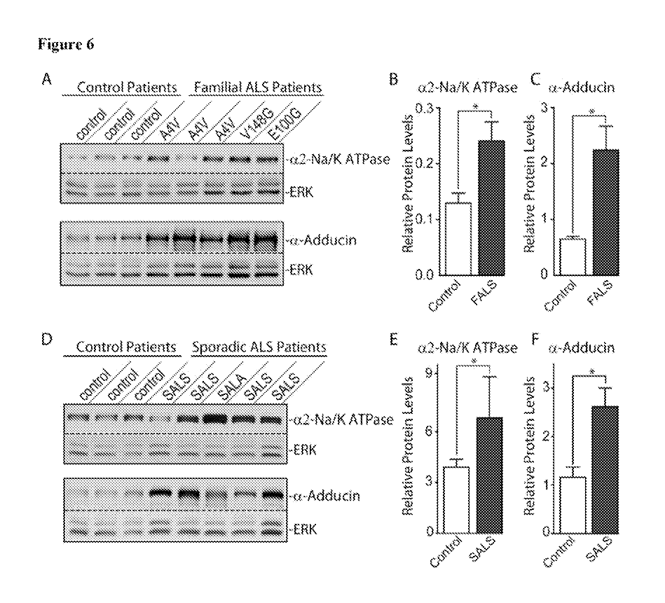

To determine the clinical relevance of the novel α2-Na/K ATPase/α-Adducin mechanism in non-cell autonomous neurodegeneration, the expression of the α2-Na/K ATPase/α-Adducin complex was characterized in spinal cord lysates from both familial ALS expressing distinct mutations in SOD1 and sporadic ALS patients and controls. Familial ALS cases demonstrated autosomal dominant inheritance. All of these patients had definite ALS by the E1 Escorial criteria, with a mean age of 42 (range of 21-65). The mean age of sporadic ALS patients was 60 (range 46-69). All patients with sporadic met modified E1 Escorial criteria for probable or definite ALS, with their spinal cords displaying gliosis, demyelination and long term motor neuron degeneration. The mean age of control patients was 64 (range 54-70). The central nervous system had normal appearance in samples of control patients. Immunoblotting of lysates of familial ALS, sporadic ALS, and control patients revealed that the levels of α2-Na/K ATPase were significantly increased in lysates of spinal cord in both familial and sporadic ALS patients as compared to controls (FIGS. 6A-6D). Likewise, the protein levels of α-Adducin were also significantly increased in spinal cord of ALS patients (FIGS. 6A-6D). Quantification of α2-Na/K ATPase and α-Adducin immunoreactivity as a continuous variable revealed that the levels of these two proteins doubled in both familial and sporadic ALS (FIGS. 6B, 6C, 6E and 6F) Together, these data indicate that the abundance of the α2-Na/K ATPase/α-Adducin complex in familial and sporadic ALS mimics the elevated levels of the complex in SOD1^{G93A} mice and may thus contribute to neurodegeneration.

### Example 6

### Therapeutic Effect of Digoxin in the Treatment of Mutant SOD1 Mice

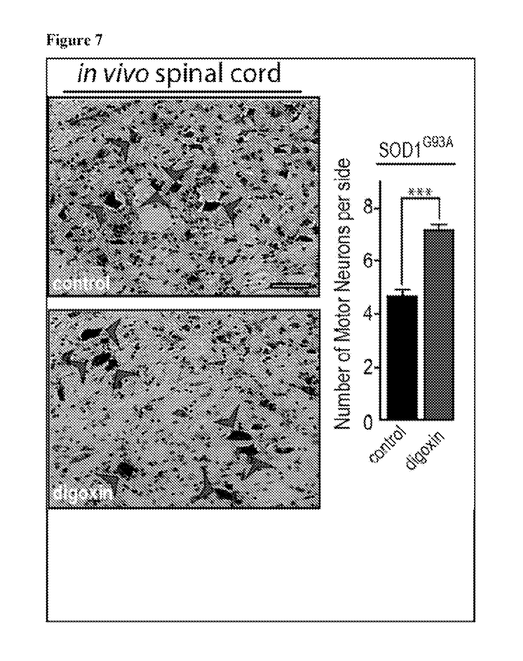

Whether the intraperitoneal injection of digoxin was neuroprotective in SOD1^{G93A} mice was tested. The blood brain barrier is modestly permeable to digoxin, leading to concentrations of 10% in the cerebrospinal fluid relative to serum (Toda et al., J. Pharm. Sci. 100:3904-3911 (2011); Liu et al., Drug. Metab. Dispos. 40:963-969 (2012)). An intraperitoneal injection of digoxin was administered at a concentration of 1 mg/kg in 80 day-old mice on a 48-hour cycle. Quantification of motor neuron survival revealed that intraperitoneal digoxin protected motor neurons from degeneration in SOD1^{G93A} mice. Control vehicle-treated SOD1^{G93A} mice toward end stage (130±5 days) harbored 4.66±0.40 motor neurons per ventral horn section (FIG. 7). By contrast, age matched digoxin-treated SOD1^{G93A} mice had 7.20±0.21 motor neurons per ventral horn section (FIG. 7). These data reveal that pharmacological inhibition of Na/K ATPase activity in vivo attenuates motor neuron degeneration in SOD1^{G93A} mice.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating a neurodegenerative disease or a neurodevelopmental disorder comprising administering to a subject an agent that inhibits α2-Na/K ATPase and/or α-Adducin.

2. The method of claim 1, wherein the disease is a neurodegenerative disease selected from the group consisting of amyotrophic lateral sclerosis (ALS), Huntington's disease, spinocerebellar ataxias, Alzheimer's disease, traumatic brain injury and Parkinson's disease or a neurodevelopmental disorder selected from the group consisting of fragile X syndrome, Down's syndrome, Rett syndrome, intellectual disability, autism, an autism spectrum disorder and Asperger syndrome.

3. The method of claim 2, wherein the neurodegenerative disease is ALS.

4. The method of claim 1, wherein the agent is selected from the group consisting of a small molecule, an interfering nucleic acid molecule specific for α2-Na/K ATPase, an antibody that binds to α2-Na/K ATPase, an isolated soluble polypeptide comprising at least 5 consecutive amino acids of the amino acid sequence encoding α-Adducin, an interfering nucleic acid molecule specific for α-Adducin, and an isolated soluble polypeptide comprising at least 5 consecutive amino acids of the amino acid sequence encoding α2-Na/K ATPase.

5. The method of claim 4 wherein the small molecule is a cardiac glycoside.

6. The method of claim 5, wherein the small molecule is selected from the group consisting of digoxin, ouabain, digitoxin, proscillaridin A, digoxigenin, gitoxin, gitoxigenin, oleandrin, butalin, cinobufagenin, UNBS1450 and lanatoside C.

7. The method of claim 6, wherein the small molecule is digoxin.

8. (canceled)

9. The method of claim 4, wherein the interfering nucleic acid molecule is an antisense molecule, an siRNA molecule, an shRNA molecule or a miRNA molecule.

10.-19. (canceled)

20. The method of claim 1, wherein the neurodegenerative disease is amyotrophic lateral sclerosis (ALS), Huntington's disease or spinocerebellar ataxias.

21.-27. (canceled)

28. A method of determining whether a test agent is a candidate therapeutic agent for the treatment of a neurodegenerative disease or a neurodevelopmental disorder, the method comprising:
a) forming a test reaction mixture comprising:
a α2-Na/K ATPase polypeptide or fragment thereof;
a α-Adducin polypeptide or fragment thereof; and
a test agent;
b) incubating the test reaction mixture under conditions conducive for the formation of a complex between the α2-Na/K ATPase polypeptide or fragment thereof and the α-Adducin polypeptide or fragment thereof; and
c) determining the amount of the complex in the test reaction mixture;
wherein a test agent that reduces the amount of the complex in the test reaction mixture compared to the amount of the complex in a control reaction mixture is a candidate therapeutic agent for the treatment of a neurodegenerative disease.

29. The method of claim 28, wherein the test agent is an antibody, a protein, a peptide or a small molecule.

30. The method of claim 28, wherein the control reaction mixture is substantially identical to the test reaction mixture except that the control reaction mixture does not comprise a test agent.

31. The method of claim 28, wherein the control reaction mixture is substantially identical to the test reaction mixture except that the control reaction mixture comprises a placebo agent instead of a test agent.

32.-44. (canceled)

45. The method of claim 28, wherein the test agent is a member of a library of test agents.

46. The method of claim 28, wherein the test agent is a small molecule.

47. A method of determining whether a subject has or is predisposed towards a neurodegenerative disease, the method comprising analyzing a cerebral spinal fluid sample from the subject to determine the expression level of α-Adducin and/or α2-Na/K ATPase in the sample, wherein elevated expression of α-Adducin and/or α2-Na/K ATPase indicates that the subject has or is predisposed towards a neurodegenerative disease.

48. The method of claim 47, wherein the neurodegenerative disease is amyotrophic lateral sclerosis (ALS), Huntington's disease, spinocerebellar ataxias, Alzheimer's disease, traumatic brain injury or Parkinson's disease.

49. The method of claim 47, wherein the neurodegenerative disease is ALS.

50. The method of claim 47, wherein the expression of α-Adducin and/or α2-Na/K ATPase in the sample is elevated if it is higher than the expression of α-Adducin and/or α2-Na/K ATPase in a control cerebral spinal fluid sample.

51.-53. (canceled)

54. The method of claim 47, further comprising administering to the subject an agent that inhibits a α-Adducin or α2-Na/K ATPase if the subject is identified as having or being predisposed towards a neurodegenerative disease.

55.-62. (canceled)
